(19) United States
(12) Patent Application Publication
(10) Pub. No.: US 2016/0304676 A1
(43) Pub. Date: Oct. 20, 2016

(54) PROCESS TO MAKE STORAGE STABLE POLYMER FORMULATIONS

(71) Applicant: DOW GLOBAL TECHNOLOGIES LLC, Midland, MI (US)

(72) Inventors: Daniel M. TRAUTH, Crystal Lake, IL (US); Qichun WAN, Midland, MI (US); Kishore K. KAR, Midland, MI (US); Justin J. WALKER, Providence, RI (US); Anthony NEUBAUER, Piscataway, NJ (US); David L. MALOTKY, Midland, MI (US)

(73) Assignee: DOW GLOBAL TECHNOLOGIES LLC, Midland, MI (US)

(21) Appl. No.: 15/102,078

(22) PCT Filed: Dec. 15, 2014
(86) PCT No.: PCT/US14/70283

§ 371 Date: Jun. 6, 2016

Related U.S. Application Data
(60) Provisional application No. 61/916,573, filed on Dec. 16, 2013.

(51) Int. Cl. C08J 3/09; A61K 8/31; A61K 8/04; A61K 8/81

(52) U.S. Cl. CPC C08J 3/092; A61K 8/8111; A61K 8/31; A61K 8/042; A61Q 19/00

(57) ABSTRACT

Disclosed is a process for making stable and consistent polymer formulations with desirable sensorial qualities.

### CROSS-REFERENCE TO RELATED APPLICATIONS

This application claims priority to U.S. Provisional Application No. 61/916,573, filed Dec. 16, 2013, the disclosure of which is hereby incorporated by reference in its entirety.

### BACKGROUND OF THE INVENTION

1. Field of the Invention

The invention relates to a process for making polymer formulations.

2. Description of Related Art

Personal care products, such as leave-on skin care, sun care, hair care, fabric care, and surface care products, require desirable aesthetics (i.e., a smooth and silky feel on the surface the product is applied). Accordingly, the personal care art has developed sensory agents, such as silicone oils, hard particles (such as poly(methyl methacrylate) (PMMA) particles and polyethylene (PE) particles), and silicone elastomer gels in order to impart good aesthetics. However, many of these sensory agents have drawbacks, like insufficient sensory performance, dry after-feel on the surface the product is applied, or relatively high cost. To overcome these drawbacks, polymer formulations have been developed. In order for polymer formulations to be commercially viable, a process to reproducibly make stable and consistent polymer formulations with desirable sensorial qualities is necessary.

### SUMMARY OF THE INVENTION

The invention relates to process to make stable and consistent polymer formulations with desirable sensorial qualities.

The invention provides a method for making a polymer formulation, the method comprising:
forming a heated mixture comprising a polymer and an oil, wherein
the temperature of the heated mixture is at least about the melting temperature of the polymer; and
cooling the mixture at a cooling rate less than about 0.7° C./minute.

These and other features and advantages of the present invention will be more fully understood from the following detailed description of the invention taken together with the claims. It is noted that the scope of the claims is defined by the recitations therein and not by the specific discussion of features and advantages set forth in the present description.

### DETAILED DESCRIPTION OF THE INVENTION

The term “oil” refers to a nonpolar chemical substance that is hydrophobic and/or lipophilic. An oil can be a hydrocarbon, having only carbon and hydrogen atoms, or have one or more heteroatoms, such as a lipid. An oil can occur naturally or can be produced synthetically.

The term “cosmetically acceptable” refers to ingredients typically used in personal care compositions. Materials that are toxic when present in the amounts typically found in personal care compositions are not contemplated as part of the present invention.

The term “polyolefin” refers to a polymer produced from the polymerization of a monomer having an olefin, also referred to as an alkene. For example, polyethylene is the polyolefin produced by polymerizing the olefin ethylene.

The term “metallocene catalyzed polyolefin” refers to polyolefins produced with a metallocene catalyst as described in U.S. Pat. Nos. 4,701,432, 5,322,728, and 5,272,236, each of which is incorporated herein by reference in its entirety. Metallocene catalyzed polyolefins are polyethylenes produced with a metallocene catalyst. Such metallocene catalyzed polyethylenes are available, e.g., from The Dow Chemical Company under the trademarks AFFINITY™ or ENGAGE™ (ethylene/octene copolymers) and from Exxon Chemical Company under the trademark EXACT™ (ethylene/butene copolymers, ethylene/hexene copolymers, or ethylene/butene/hexene terpolymers). Metallocene catalyzed polyolefins can be one of ethylene/octene copolymers, ethylene/butene copolymers, ethylene/hexene copolymers, ethylene/propylene or ethylene/butene/hexene terpolymers. Metallocene catalyzed polyolefins can also be propylene/alpha-olefin copolymers, as described in U.S. Pat. Nos. 6,960,635 and 6,525,157, each of which is incorporated herein by reference in its entirety. Propylene/alpha-olefin copolymers are commercially available from The Dow Chemical Company, under the trademark VERSIFY™ or from ExxonMobil Chemical Company, under the trademark VISTAMAXX™. Other desirable polyolefins are sold by The Dow Chemical Company under the trademarks AMPLITY™, ATTANE™, INFUSE™, NORDEL™, and VLDPE™.

The term “melt index” is synonymous with “melt flow index” and “melt flow rate”, and refers to a measurement of the ease of which a thermoplastic polymer flows when melted. It is defined as a mass of polymer, in grams, flowing through a capillary of a specific diameter and length in ten minutes when pressure and temperature are applied. Melt index is proportional to molecular weight and is inversely proportional to viscosity.

The term “storage stable” refers to products that do not substantially change in composition during storage at ambient temperature ±20° C., for the duration of their expected product lifetime. An unstable polymer formulation has an unstable viscosity, e.g., where the polymer formulation becomes substantially solid or where the oil and the polymer in the polymer formulation separate.

The invention provides a method for making a polymer formulation, the method comprising:
forming a heated mixture comprising a polymer and an oil, wherein
the temperature of the heated mixture is at least about the melting temperature of the polymer; and
cooling the mixture at a cooling rate less than about 0.7° C./minute.

In some embodiments the method further includes cooling the heated mixture to a temperature between about 50° C. to about 80° C. The heated mixture can also be cooled to a temperature between about 60° C. to about 70° C. In some instances the method further includes transferring the cooled mixture to a container. Transfer can occur when the temperature of the cooled mixture is between about 50° C. to about 80° C., or between about 60° C. to about 70° C.

In some embodiments, the polymer comprises one or more metallocene catalyzed polyolefins. In some embodiments, the polymer can include at least one polyolefin with a density greater than 0.90 g/cm³, and at least one metallocene catalyzed polyolefin with a density lesser than or equal to 0.90 g/cm³. In some embodiments, the density of the metallocene catalyzed polyolefin with a density greater than 0.90 g/cm³ has a density between about 0.90 g/cm³ and about 0.95 g/cm³, or between about 0.91 g/cm³ and about 0.93 g/cm³. In other embodiments, the density of the metallocene catalyzed polyolefin with a density less than 0.90 g/cm³ has a density between about 0.80 g/cm³ and about 0.89 g/cm³, or between about 0.85 g/cm³ and about 0.89 g/cm³.

In some embodiments, the average melt index is between about 0.8 and 500. In some embodiments, the average melt index of the polyolefin is greater than 7. In other embodiments, the average melt index is between 1 and 20.

Table 1 contains a list of commercially available metallocene catalyzed polyolefins with their average melt index and density.

TABLE 1
Commercially available metallocene catalyzed polyolefins.

| | | Density |
|---|---|---|
| Polyolefin Name | Melt Index | (g/cm³) |
| AFFINITY GA 1950 | 500 | 0.874 |
| AFFINITY PL1840G | 1 | 0.909 |
| AMPLIFY EA 103 | 21 | 0.930 |
| AMPLIFY GR 202 | 8 | 0.930 |
| ATTANE 4203 | 0.8 | 0.905 |
| ATTANE 4404G | 4 | 0.904 |
| ENGAGE 8100 | 1 | 0.870 |
| ENGAGE 8130 | 13 | 0.863 |
| ENGAGE 8200 | 5 | 0.870 |
| ENGAGE 8402 | 30 | 0.902 |
| LDPE 4016 | 16 | 0.916 |
| LDPE 640I | 2 | 0.920 |
| LDPE 955I | 35 | 0.923 |
| VERSIFY 2200 | 2 | 0.876 |
| VERSIFY 3200 | 8 | 0.876 |
| VERSIFY 4200 | 25 | 0.876 |

In some embodiments, the polyolefin is free of, or substantially free of, ethylene acrylic copolymer. Copolymerizing ethylene with acrylic acid yields ethylene-acrylic acid (EAA) copolymers, which are known for use in personal care compositions. However, their relatively low pH and low surfactant levels are not compatible with some skin care compositions.

The polyolefin can include a mixture of at least one metallocene catalyzed polyolefin with a density greater than 0.90 g/cm³ and at least one metallocene catalyzed polyolefin with a density lesser than or equal to 0.90 g/cm³. The mixture can have a weight ratio of about 95:1 to about 1:95. In some embodiments, the weight ratio can be 1:1, 1.5:1, 2:1 or 3:1. In certain instances, the weight ratio can be between about 3:1 and about 1:1.

The oil can be any oil capable of use in a polymer formulation on human skin. The oil should be non-toxic in regard to the amount present in the formulation, the amount applied to the skin, the duration of contact time, and the cumulative daily exposure to the skin. The oil can be unsaturated or saturated, and can include other functionality such as ester, alcohol, and carboxylic acid groups. In some embodiments, the oil is a lipid, such as a fatty acid, or a mixture of lipids. For example, the oil can be jojoba oil. In some embodiments, the oil is a natural oil that is produced by a plant, animal, or other organisms through natural metabolic processes. Such oils include vegetable oils, nut oils, citrus oils, melon oils, lipids, fatty acids, triglycerides, polyols and beeswax. In other embodiments, the oil is a synthetic oil. A synthetic oils can be manufactured using chemically modified petroleum components or other raw materials. In some instances, the synthetic oil can include naturally occurring components combined to form a non-naturally occurring mixture. In other embodiments, the oil can be a petroleum oil, such as mineral oil or liquid paraffin, or a silicone-based oil. In some embodiments, the oil can be a C₁₄ to C₂₂ hydrocarbon oil. Acceptable hydrocarbon oils can have straight carbon chains, such as tetracosane, or branched carbon chains, such as isohexadecane. In some embodiments, the oil can be a cosmetically acceptable oil. The oil can also be a mixture of any of the oils described herein.

The amount of the polymer in the heated mixture can be about 13 wt. % to about 17 wt. % of the heated mixture, or about 14 wt. % to about 16 wt. % of the mixture. In some instances, using a polymer concentration above about 16 wt. % can provide a product with a high viscosity. In other instances, using a polymer concentration below about 14 wt. % can provide an unstable mixture where upon cooling the mixture the polymer separates from the oil. In certain instances, the polymer concentration is about 15 wt. % by weight.

The temperature of the heated mixture should be sufficient to melt the polymer and/or allow it to substantially dissolve in the oil. Most polymers used in the method have a melting point of less than or equal to about 105° C., so in most embodiments, the temperature of the mixture is equal to or greater than about 110° C. But if the polymer has a lower melting point, the temperature of the mixture can be adjusted accordingly. The temperature is usually maintained at about 5° C. to about 50° C. above the melting point of the polymer. In some instances, the temperature is about 5° C. to about 20° C., or about 5° C. to about 15° C. above the melting temperature of the polymer. In most embodiments, the temperature of the mixture is less than about 150° C.

In some embodiments, the heated mixture is formed by adding the polymer to a pre-heated, oil. The temperature of the pre-heated oil is less than the flash point of the oil. The flash point of a substance is the lowest temperature at which it can vaporize to form an ignitable mixture in air. When the temperature of the mixture is equal to or greater than the flash point of the oil, special precaution may be necessary to avoid combustion of the oil. Because combustion requires oxygen, the probability of combustion can be limited by performing the method under oxygen-free conditions, such as under nitrogen or argon.

In some embodiments, the temperature of the pre-heated oil is greater than the highest melting temperature of the polymer, and less than the flash point of the oil.

The temperature of the heated mixture can be obtained before or after the polymer is added to the oil. In some embodiments the polymer is added to the oil at ambient temperature and the mixture is heated. In other embodiments, the oil is preheated before the addition of the polymer. The preheated temperature can be equal to or greater than the temperature of the heated mixture. That is, the polymer can be added to an oil that has been preheated to a temperature that is equal to or greater than the target temperature of the heated mixture. In some embodiments, the pre-heated oil is heated to a temperature greater than or equal to about 110° C. Alternatively, the oil can be preheated to a temperature between ambient temperature and the target temperature before the polymer is added. After addition is complete, the mixture is then heated to the target temperature. Regardless of the method used to dissolve the polymer, the temperature of the heated mixture should not fall below the melting point of the added polymer. Doing so can result in the potential agglomeration of the polymer from the mixture.

Once the heated mixture reaches the targeted temperature, the mixture is mixed for about 10 to about 120 minutes. In some embodiments, the mixture is stirred for about 30 to about 60 minutes, while in other instances the mixture is stirred for about 60 min. Care should be taken to maintain the temperature of the heated mixture within about ±20° C. of the target temperature. In some embodiments, the temperature of the heated mixture is maintained within about ±10° C. of the target temperature. In some instances, if the temperature of the heated mixture falls below the melting point of the polymer, polymer agglomeration can occur.

Mixing should be performed so that the polymer is able to be completely dissipated throughout the oil. Depending on the temperature, the mixture can become viscous, and proper care should be taken to aid the dissolution of the polymer by increasing shear limit the amount of polymer that may cool on the sides of the reaction vessel. Therefore, in some embodiments, the method is performed in a jacketed vessel with one or more mechanical agitators. A jacketed flask allows for even heating and cooling of the mixture, and mechanical agitators are able to overcome the viscosity of the mixture. In some embodiments, agitation is performed with two independently driven agitators: 1) a paravisc agitator moves the material away from the wall and bottom of the mixer and 2) a viscoprop agitator moves the material in a downward motion in the center of the reactor. In some embodiments the agitators can be set to rotate in opposite directions. This method of mixing can provide the shear needed to dissolve the polymer and maintain a homogeneous consistency within the reaction vessel. In some embodiments, the mixers are co-axial mixers consisting of a proximity impeller and an open impeller. Other suitable mixers include planetary mixers and single or multiple high shear mixers. In some embodiments, the mixer is a co-axial mixer including a proximity impeller, such as a paravisc, and an open impeller, such as a viscoprop.

Once the polymer is completely dissolved in the oil, the mixture can be cooled. The cooling rate affects the characteristics of the resulting polymer formulation. If the mixture is cooled too fast, the formulation can be too viscous. In some embodiments, the cooling rate is less than or equal to about 0.7 degrees ° C./minute. Slow cooling rates (i.e., less than about 0.1° C./min) do not appear to have a negative affect the properties of the resulting formulation, but they can inhibit the efficiency of the process by requiring long cooling times. In some embodiments, the cooling rate is about 0.35° C./minute. In other instances, the cooling rate is about 0.20° C./minute.

In some embodiments, the method further comprises cooling the heated mixture to a specific temperature, typically between about 50° C. and about 80° C. In other embodiments, the mixture is cooled to between about 60° C. and 70° C. Once the mixture reaches this temperature, it can be discharged from the reaction vessel. In some situations the mixture is discharged into a sample container, but discharging can be accomplished by transferring the formulation into any container or number of containers. The discharge temperature can have an effect on the viscosity of the formulation. If the discharge temperature is too high, for example, above 90° C., the polymer can crash out of the oil, which can transform the formulation into a two-phase, rubbery mixture. If the discharge temperature is too low, the formulation can become too thick and make it difficult to discharge the formulation from the reaction vessel. In some embodiments, these issues can usually be avoided if the discharge temperature of the mixture is between about 60° C. to about 70° C.

The method provides a formulation having a viscosity of between about 100,000 and about 1,000,000 cP. In some embodiments, the viscosity of the formulation can be between about 400,000 and about 500,000 cP. In certain embodiments, the formulation is a gel.

Thus, in certain aspects, the product of the methods is a polyolefin gel. Also, a gel made previously using the methods of the invention may be reprocessed using the same methods. For example, in cases where the obtained gel does not have the desired characteristics and/or properties, the gel can replace the heated mixture used in the method. The cooling rate, discharge temperature, mixture temperature, polyolefin and polyolefin concentration can then be varied to provide the desired gel.

### EXAMPLES

### Example 1

### Preparation of a Polyolefin Gel

Batches 1-7 were run in a 50 Liter Ekato Unimix SRT-50 Dual Action Mixer that is jacketed so the mixer contents can be heated or cooled. Two motors independently drive the two agitators: 1) a paravisc agitator moves the material away from the wall and bottom of the mixer and 2) a viscoprop agitator moves the material in a downward motion in the center of the reactor. The agitators are typically set to rotate in opposite directions in order to develop shear and maintain a homogeneous consistency in the mixer.

The procedure was as follows: Mineral oil was added to the reaction vessel and mixed as it was heated to 120° C. When the mineral reached 120° C., the polyolefin was added. During addition, mixing was continued and the reaction temperature was maintained between 110 and 120° C. The mixture was maintained at 120° C. for 1 hour, and then allowed to cool to its discharge temperature. The mixture was discharged from the reaction vessel into sample containers, and the viscosity was measured.

Table 2 shows the parameters of each batch and the viscosity of the resulting polyolefin gel. A positive mixer speed indicates clockwise rotation (downwards) and a negative mixer speed indicates a counter clockwise rotation (upwards). The maximum speed for the paravisc was 120 rpm and the viscoprop was 350 rpm. Batch 6R was run using the material prepared in Batch 6. All runs used a 1:1 mixture of AFFINITY GA 1950 and AFFINITY GA 1840 polyolefin beads. Viscosity was measured with a Brookfield viscometer, Helipath D/#96 spindle, 5 rpm, at 25° C. A viscosity of “NM” means that the viscosity was not measured, and a “variable” viscosity means that the viscosity of the sample was not consistent. Viscosity measurements with a “I” correspond to samples discharged from the reaction vessel at two different temperatures.

TABLE 2
Reaction Parameters.

| | | Mix- | Cooling | | | | |
|---|---|---|---|---|---|---|---|
| | Solids | ture | rate | Para- | Visco- | Discharge | Vis- |
| Batch | (wt. | Temp | (° C./ | visc | prop | Temp | cosity |
| # | %) | (° C.) | hr) | (rpm) | (rpm) | (° C.) | (cP) |
| 1 | 16.0 | 120 | 12.5 | −90 | 262 | 70 | NM |
| 2 | 15.0 | 120 | 12.5 | −90 | 262 | 70 | 632,000 |
| 3 | 15.0 | 120 | 12.5 | −30 | 100 | 70/60 | Variable/ |
| | | | | | | | 466,000 |
| 4 | 15.0 | 120 | 15 | −60 | 175 | 60 | 427,000 |
| 5 | 15.0 | 120 | 15 | −30 | 100 | 60 | 472,000 |
| 6 | 15.0 | 120 | 40 | −30 | 100 | 60 | NM |
| 6R | 15.0 | 95 | 20 | −30 | 100 | 60 | 687,000 |
| 7 | 13.8 | 120 | 15 | −30 | 100 | 70/60 | 490,000/ |
| | | | | | | | 362,000 |

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

1. A method for making a polymer formulation, comprising:
forming a heated mixture comprising a polymer and an oil, wherein the temperature of the heated mixture is at least about the melting temperature of the polymer;
cooling the mixture at a cooling rate less than or equal to about 0.7° C./minute.

2. The method of claim 1, further comprising cooling the heated mixture to a temperature between about 50° C. to about 80° C.

3. The method of claim 1, further comprising transferring the cooled mixture to one or more containers when the temperature of the cooled mixture is between about 50° C. to about 80° C.

4. The method of claim 3, wherein the temperature of the cooled mixture is about 60° C. to about 70° C.

5. The method of claim 1, wherein the polymer comprises at least one metallocene catalyzed polyolefin with a density greater than 0.90 g/cm³, and at least one metallocene catalyzed polyolefin with a density lesser than or equal to 0.90 g/cm³.

6. The method of claim 5, wherein the weight ratio of at least one metallocene catalyzed polyolefin with a density greater than 0.90 g/cm³ and at least one metallocene catalyzed polyolefin with a density lesser than or equal to 0.90 g/cm³ is about 3:1 to about 1:1.

7. The method of claim 1, wherein the average melt index of the polymer is between about 1 and about 20.

8. The method of claim 1, wherein the oil is a natural oil comprising one or more fatty acids.

9. The method of claim 1, wherein the oil is a synthetic oil.

10. The method of claim 1, wherein the oil is C₁₄ to C₂₂ hydrocarbon oil.

11. The method of claim 1, wherein the heated mixture is formed by adding the polymer to an oil at ambient temperature and heating the resulting mixture.

12. The method of claim 1, wherein the heated mixture is formed by adding the polymer to a pre-heated oil and heating the resulting mixture.

13. The method of claim 12, wherein the pre-heated oil is heated to a temperature greater than or equal to about 110° C.

14. The method of claim 1, wherein the cooling rate is about 0.35° C./minute.

15. The method of claim 1, wherein the cooling rate is about 0.20° C./minute.

16. A method for making a polyolefin formulation, comprising:
forming a heated mixture comprising a polyolefin and an oil, wherein the temperature of the heated mixture is at least about the melting temperature of the polymer;
cooling the mixture at a cooling rate less than or equal to about 0.7° C./minute,
wherein the polyolefin comprises at least one metallocene catalyzed polyolefin with a density greater than 0.90 g/cm³, and at least one metallocene catalyzed polyolefin with a density lesser than or equal to 0.90 g/cm³.

17. A method according to claim 1, wherein the polymer comprises an ethylene/octene copolymer, ethylene/butene copolymer, ethylene/hexene copolymer, ethylene/butene/hexene terpolymer, ethylene/hexene copolymer, ethylene/propylene/hexene copolymer, ethylene/butene/hexene terpolymer, or propylene/alpha-olefin copolymer.

18. A method for making a polymer formulation having a viscosity of between about 100,000 and about 1,000,000 cP, comprising:
forming a heated mixture comprising a polymer and an oil, wherein the temperature of the heated mixture is at least about the melting temperature of the polymer;
cooling the mixture at a cooling rate less than or equal to about 0.7° C./minute.

19. A method according to claim 18, wherein the polymer formulation has a viscosity of between about 400,000 and about 500,000 cP.

20. A method according to claim 18, wherein the polymer formulation is a gel.
